(19) United States
(12) Patent Application Publication
(10) Pub. No.: US 2023/0095495 A1
(43) Pub. Date: Mar. 30, 2023

(54) SKIN CONDITIONING COMPOSITION WITH HIGH CONCENTRATION OF UREA TO BOOST THE SKINCARE ACTIVES

(71) Applicant: L'OREAL, Paris (FR)

(72) Inventors: Rebecca CHEN, Princeton, NJ (US); Yixin YANG, Union, NJ (US); Anne-Laure BERNARD, New York, NY (US)

(73) Assignee: L'OREAL, Paris (FR)

(21) Appl. No.: 17/953,392

(22) Filed: Sep. 27, 2022

Related U.S. Application Data
(60) Provisional application No. 63/250,577, filed on Sep. 30, 2021.

(30) Foreign Application Priority Data
Dec. 15, 2021 (FR) 2113553

(51) Int. Cl. A61K 8/42; A61K 8/44; A61K 8/36; A61K 8/81; A61Q 19/08

(52) U.S. Cl. CPC A61K 8/42; A61K 8/44; A61K 8/36; A61K 8/81; A61Q 19/08; A61K 2800/874

(57) ABSTRACT

A method for improving the efficacy of a skin care active includes applying, in any order, a first composition that includes a skin conditioning composition in carrier system having a water phase having: urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH is less than about 7; and a second composition that includes a complementary composition including one or more skin actives not present in the skin conditioning composition. The complementary composition is a leave on or rinse of skin peel composition including at least a hydroxy acid. The compositions may be provided as an article of manufacture with separately contained compositions.

### RELATED APPLICATIONS

This application claims the benefit of, and priority to, U.S. Provisional Patent Application Ser. No. 63/250,577, filed on Sep. 30, 2021, entitled SKIN CONDITIONING COMPOSITION WITH HIGH CONCENTRATION OF UREA TO BOOST THE SKIN CARE ACTIVES, as well as French Application No. FR2113553, filed on Dec. 15, 2021, the entirety of which are incorporated by reference herein.

### FIELD OF TECHNOLOGY

The present disclosure is in the field of cosmetic compositions for topical application to keratinous tissue, particularly compositions that include urea in high amounts together with actives for which the urea may provide stabilization and or boosted efficacy.

### BACKGROUND

Many cosmetic compositions are provided with select actives that provide any number of benefits, for example, including but not limited to skin desquamation (peeling). Urea is a commonly used ingredient as a humectant. High concentrations of urea for skin care are desirable for the humectant properties, including as a means to promote the stratum corneum to swell, and for stimulating skin desquamation. Urea is found in many compositions that include other skin actives. Various compositions that may include high concentrations of urea for skin care are thus desirable for the humectant properties. Such skin care compositions would beneficially include urea or a derivative thereof in a range from about 20% to 40% by weight of the composition. However, it is well known that topical preparations that contain high concentrations of urea present many challenges One challenge relates to the tendency of urea to undergo a continuous hydrolysis reaction that leads to pH increase, which can result in an increase from a pH of about 7 up to a pH of 9 or greater over the course of storage. Such pH values at or greater than a pH of 8 are much higher than that of the natural skin, which is about 5.5, and compositions with a pH in this range would likely cause skin irritation when applied. Thus, such high resultant pH renders most formulations unsuitable for use on skin, particularly in water-based compositions which don't provide protection through barrier forming oils and butters. This is particularly an issue for water-based leave-on compositions. Yet another challenge is the tendency of urea to crystalize at high concentrations and at low temperature over time. This crystallization would be expected to reduce the amount of active urea available in the usable composition, thus diminishing the overall desired efficacy of the product. A further challenge with high urea content is whitening of the skin which is not aesthetically pleasing to many consumers.

The inventors provide here a cosmetic skin care composition comprising a skin conditioning composition that is used in some embodiments for skin rejuvenation that includes use of the skin conditioning composition followed by use of a secondary complementary composition. In some embodiments the inventors have overcome the foregoing challenges and provide a water-based skin conditioning composition that supports and, in some embodiments, boosts the activity of skin actives. In some embodiments the skin conditioning composition is stable from pH increase and crystallization to maintain a high amount of urea for achieving efficacy as a humectant, for promoting the stratum corneum to swell, and for stimulating skin desquamation, among other possible benefits.

### BRIEF SUMMARY OF THE INVENTION

This summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In accordance with various embodiments, provided is a home use skin rejuvenation system with high urea content in different formats selected from a water toner, a gel, a mask, a micellar water, a cream, a lotion, a cleanser, or a make-up remover. According to some embodiments, an inventive skin conditioning composition may be used alone or as first step prior to application of a secondary complementary composition comprising one or more actives, such as a hydroxy acid or other water soluble actives, to boost the efficacy of the active or actives, for example for skin rejuvenation.

In another embodiment, provided are methods of use of the skin conditioning composition. In some embodiments, a method of use includes application of the skin conditioning composition as a leave on for providing moisture retention benefits. In some embodiments, a method of use includes application of the skin conditioning composition as a leave on, followed by application, of a complementary composition, such as but not limited to a complementary composition containing one or more actives that can complement or supplement the benefits of the skin conditioning composition.

In some embodiments, the disclosure provides a skin rejuvenation system (or kit) employing the skin conditioning composition and one or more complementary compositions comprising one or more actives, such as a hydroxy acid or other water soluble actives. In some embodiments, the disclosure provides a regimen employing the skin conditioning composition.

In various embodiments of use of the skin conditioning composition and skin rejuvenation system, the regimen includes application, in one, two, three or more application steps of a first composition comprising at least one of the skin conditioning composition and a complementary composition, and a second composition comprising at least one of the skin conditioning composition and a complementary composition. In some embodiments, the first and second compositions are different. In some embodiments, the first and second compositions are the same. Accordingly, in some embodiments, the first composition comprises the skin conditioning composition, and the second composition comprises the complementary composition. And in other embodiments, the first composition comprises the complementary composition, and the second composition comprises the skin conditioning composition. Application may include three or more sequential steps where first and second compositions are applied in any order.

In some embodiments after application of the complementary composition, both compositions are either left on the skin or may be removed by rinsing. In some embodiments, the application of one or both may include use of an implement such as a brush.

In another aspect, the present disclosure provides a method of contacting a portion of skin with skin conditioning composition with or without an implement or an applicator. The methods may further include application of a complementary composition, before or after application of the skin conditioning composition.

In some particular embodiments, provided is a method improving the efficacy of a skin care active, comprising:

a) applying to a region of skin a first composition comprising a skin conditioning composition comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH is less than about 7;
b) applying to the region of skin a second composition comprising complementary composition comprising one or more skin actives not present in the skin conditioning composition.

In accordance with some embodiments, the steps of the method include (a) and (b) in any order.

In accordance with some embodiments, each of the compositions is aqueous and is applied in liquid form.

In accordance with some embodiments, the first composition is one of a water based toner or a mask.

In accordance with some embodiments, the first composition is a water based toner that is applied first followed by the second composition without prior removal of the first composition.

In accordance with some embodiments, the first composition is a water based mask that is applied first followed by the second composition, the method further comprising a step that includes rinsing to remove the first composition prior to application of the second composition.

In accordance with some embodiments, one or both of the first and second compositions is coupled to an application surface of an applicator, the application surface selected from the group consisting of a bristle surface of a brush, a roller surface of a rolling applicator, and a blade surface of a spatula.

In other embodiments, provided is an article of manufacture for providing a skin skin rejuvenation system for improving the efficacy of a skin care active, the article of manufacture comprising: at least first and second separately contained compositions

a. the first composition comprising a skin conditioning composition comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH is less than about 7, and
b. second composition comprising complementary composition comprising one or more skin actives not present in the skin conditioning composition.

In accordance with some embodiments, the first and second compositions are one of (i) contained to be mixable prior to application to skin, and (ii) contained to be applied separately, in any order.

In accordance with any of the embodiments disclosed herein, one or both compositions may be coupled to an application surface of an applicator. In accordance with any of the embodiments disclosed herein, the application surface may be a surface of a mask configured to conform to a face of a wearer. In accordance with any of the embodiments disclosed herein, the application surface may be selected from the group consisting of a bristle surface of a brush, a roller surface of a rolling applicator, and a blade surface of a spatula. In accordance with any of the embodiments disclosed herein, the skincare formulation may be disposed in a first compartment of a sachet and the application surface may be a surface of a second compartment of the sachet, and wherein the first compartment and the second compartment are fluidically separated by a breakable barrier.

Other features and advantages of the present invention will be apparent from the following more detailed description, taken in conjunction with the accompanying drawings which illustrate, by way of example, the principles of the invention.

The detailed description set forth below in connection with the appended drawings, where like numerals reference like elements, is intended as a description of various embodiments of the disclosed subject matter and is not intended to represent the only embodiments. The embodiments described in this disclosure are provided merely as examples or illustrations and should not be construed as preferred or advantageous over other embodiments. The illustrative examples provided herein are not intended to be exhaustive or to limit the claimed subject matter to the exact forms disclosed.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE DRAWINGS

FIG. 1 is a bar graph that shows stratum corneum test results demonstrating the comparative efficacy of the inventive composition used according to the inventive methods as compared with controls;

FIG. 2 is a bar graph that shows stratum corneum test results demonstrating the comparative efficacy of the inventive composition at different concentrations of urea and used according to the inventive methods as compared with controls; and

FIG. 3 is a bar graph that shows stratum corneum test results demonstrating the comparative efficacy of the inventive composition employing different forms of urea and used according to the inventive methods as compared with controls.

Aspects and many of the attendant advantages of the claimed subject matter will become more readily appreciated as the same become better understood by reference to the following detailed description, when taken in conjunction with the accompanying drawings.

The detailed description set forth below in connection with the appended drawings, where like numerals reference like elements, is intended as a description of various embodiments of the disclosed subject matter and is not intended to represent the only embodiments. The embodiments described in this disclosure are provided merely as examples or illustrations and should not be construed as preferred or advantageous over other embodiments. The illustrative examples provided herein are not intended to be exhaustive or to limit the claimed subject matter to the exact forms disclosed.

### DETAILED DESCRIPTION OF THE INVENTION

The skin conditioning composition of the present invention may be used for the production of cosmetic preparations, or dermatological preparations, more particularly topical treatment preparations, that may be formulated as single-phase water-based solution compositions, cosmetic serums, or aerosols, for example. Topical application to a surface may be a surface such as the mucus membrane or the skin, for example.

In various embodiments, the disclosure provides a cosmetic skin care composition comprising a skin conditioning composition with high urea content in different formats selected from a water toner, a gel, a mask, a micellar water, a cream, a lotion, a cleanser, or a make-up remover. According to some embodiments, an inventive skin conditioning composition may be used alone or as a first step prior to application of a secondary or complementary composition comprising one or more actives, such as a hydroxy acid or other water soluble actives. According to some such embodiments, the inventive skin conditioning composition my boost the efficacy of the active or actives, for example for skin rejuvenation or conditioning.

As used herein, the terms “skin conditioning” and “skin rejuvenation” in reference to the inventive skin conditioning composition means acting as a humectant as a means to promote the stratum corneum to swell, and for stimulating skin desquamation. In some embodiments, including as exemplified herein, the skin conditioning composition is especially useful according to methods for rejuvenation including application of the skin conditioning composition to promote the stratum corneum of skin to swell to thereby stimulate the skin to be responsive to a secondary treatment, for example, exfoliation.

“Cosmetically acceptable” means compatible with any keratinous substrate. For example, “cosmetically acceptable carrier” means a carrier that is compatible with any keratinous substrate.

The term “stable” as used in reference to the structural or solubility state of the ingredients of the skin conditioning composition means that the skin conditioning composition lacks crystal formation that is visible to the naked eye, and does not demonstrate visually perceptible crystal formation over a period of time, for example for one, two or four or five days, or for a period of one or more weeks, and for example up to eight (8) weeks, and wherein when crystals are visualized at a temperature at or below 4° C., such crystals disappear with or without mild agitation within 24 hours of reaching an ambient temperature from about 25° C. up to about 37° C. and after freeze thaw cycles from −20° C. through 20° C. The term “stable” as used in reference to the pH the skin conditioning composition means that the pH does not increase appreciably over time, for example as described in the examples herein.

The term “leave-on” as used herein refers to a composition which is applied and is not removed by wiping or rinsing with water.

The disclosure provides a home use skin conditioning composition with high urea content in different formats selected from a water toner, a gel, a mask, a micellar water, a cream, a lotion, a cleanser, or a make-up remover. The skin conditioning composition is used as first step prior to application of a complementary composition comprising one or more actives, such as a hydroxy acid or other water soluble actives, to boost the efficacy of the active or actives, for example for skin rejuvenation.

Applicants have unexpectedly found that use of a skin conditioning composition that demonstrates structural and pH stability for periods of up to about 8 weeks under temperature conditions that range from 4° C. up to 45° C. and through freeze thaw cycles from −20° C. through 20° C. The stabilities are characterized as the absence of any or sustained visible crystallization and the stabilization of pH at or under a pH of about 7, and in some particular embodiments that can include greater than 20% and up to at least 40% urea. In some embodiments, the composition can surprisingly improve the efficacy of a second complimentary composition that includes at least one skin active. These benefits are accomplished wherein stability of the skin conditioning composition is characterized as the absence of any or sustained visible crystallization and the stabilization of pH at or under a pH of about 7 and includes from about or greater than 20% and up to at least 40% urea. Examples provided herein demonstrate these and other aspects of the unexpected properties of the invention.

In accordance with the disclosure, the invention also provides methods of use of the skin conditioning composition. In some embodiments, a method of use includes application of the skin conditioning composition as a leave on for providing moisture retention benefits. In some embodiments, a method of use includes application of the skin conditioning composition as a leave on, followed by application, of a complementary composition, such as but not limited to a complementary composition containing one or more actives that can complement or supplement the benefits of the skin conditioning composition.

In some embodiments, the disclosure provides a skin rejuvenation system (or kit) employing the skin conditioning composition and one or more complementary compositions comprising one or more actives, such as a hydroxy acid or other water soluble actives. In some embodiments, the disclosure provides a regimen employing the skin conditioning composition.

In various embodiments of use of the skin conditioning composition and skin rejuvenation system, the regimen includes application, in one, two, three or more application steps of a first composition comprising at least one of the skin conditioning composition and a complementary composition, and a second composition comprising at least one of the skin conditioning composition and a complementary composition. In some embodiments, the first and second compositions are different. In some embodiments, the first and second compositions are the same. Accordingly, in some embodiments, the first composition comprises the skin conditioning composition, and the second composition comprises the complementary composition. And in other embodiments, the first composition comprises the complementary composition, and the second composition comprises the skin conditioning composition. Application may include three or more sequential steps where first and second compositions are applied in any order.

In some embodiments after application of the complementary composition, both compositions are either left on the skin or may be removed by rinsing. In some embodiments, the application of one or both may include use of an implement such as a brush.

### Skin Conditioning Composition

In various embodiments, the skin conditioning composition is formed by providing the carrier system having a water-phase that comprises water. In various embodiments, the carrier system may further comprise one more additional ingredients, each present in the water phase or in another phase, such as an oily phase. In various embodiments, the one or more additional ingredients includes one or more of an additional ingredient selected from the group consisting of a skin active compound, humectant in addition to any glycol, a preservative, a surfactant, a thickener, and combinations thereof. In some embodiments, the skin conditioning composition may include one or more of any one of the additional ingredients. In some embodiments, the skin conditioning composition may also include fragrances, pH adjusters, other cosmetically acceptable additives, or a combination thereof.

The skin conditioning composition may be any suitable form, such as, but not limited to, a single phase solution comprising the water phase, a multi-phase solution comprising at least one water phase, an emulsion comprising a water phase and an oily phase, such as, but not limited to, a water-in-oil emulsion, an oil-in-water emulsion, or a silicone-in-water emulsion. The skin conditioning composition may be provided in a product form for application to keratinous tissue, such as, but not limited to, a skin care product or a hair care product, and may be a product form such as, but not limited to, a micellar water, a cream or lotion, a toner, a cleanser, or a make-up remover. The skin conditioning composition in any of the product forms may be either a leave-on or a rinse-off formulation. In certain embodiments, as exemplified herein, the skin conditioning composition is a leave-on formulation.

As further described herein below, the skin conditioning composition includes a carrier system having a water phase, the water phase including urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH of the skin conditioning composition is less than about 7. The skin conditioning composition carrier system may also include one more additional ingredients, each present in the water phase or in a second phase.

### Urea

In accordance with the various embodiments, the skin conditioning composition includes urea present in an amount sufficient to enhance penetration of the active ingredient(s).

In some embodiments, urea may be provided as one of urea, or a urea derivative, for example, hydroxyethyl urea (HEU), wherein urea has a formula (I) and HEU has a formula (II) as shown below.

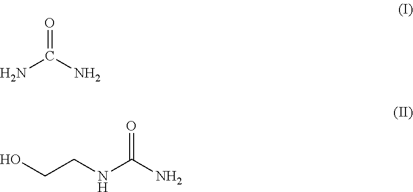

Molecular weight information for each of these is shown in Table 1.

TABLE 1

| | Molecular | | Mol/L* |
|---|---|---|---|
| | Weight | wt % | (at 1 g/L) |
| HEU | 104 | 40% | 3.85 |
| Urea | 60 | 40% | 6.67 |
| HEU | 104 | 23% | 2.21 |
| Urea | 60 | 23% | 3.88 |

Generally, a urea or a derivative thereof is selected for its beneficial effects to the skin, which effect is to be enhanced by exposure to urea of the viable epidermis and/or dermis, which underlies the stratum corneum, wherein a urea derivative can be described by the general formula (III):

R¹R²N—C(═O)—NR³R⁴ (III)

wherein R1, R2, R3 and R4 each represent, independently of each other, a hydrogen atom, a C1-C4 alkyl group or a C2-C6 hydroxyalkyl group which optionally contains from 1 to 5 hydroxyl groups, where at least one of the radicals R1 to R4 represents a hydroxyalkyl group, and their salts, their solvates and their isomers in a composition containing a physiologically acceptable medium, as agent intended for stimulating desquamation of the skin and/or the mucous membranes and/or the superficial body growths. As described in U.S. Pat. No. 7,990,08, such urea derivatives have a remarkable effect on several mechanisms involved in desquamation, thus allowing a complete and natural action of stimulating this process, and the application in all cases where it is desirable to promote and/or accelerate the removal of the dead layers of the epidermis. For compounds of formula (III):

Preferably, R, denotes a C2-C6 hydroxyl group, and R2, R3 and R4 denote, independently of each other, a hydrogen atom or a C1-C4 alkyl group;

Preferably, R1 denotes a C2-C6 hydroxyalkyl group comprising from 1 to 5 hydroxyl groups, in particular 1 hydroxyl group, and R2, R3 and R4 denote a hydrogen atom;

More preferably, R1 denotes a C2-C4 hydroxyalkyl group comprising 1 hydroxyl group, and R2, R3 and R4 denote a hydrogen atom.

Among the alkyl groups, there may be mentioned in particular methyl, ethyl, n-propyl, isopropyl, n-butyl, isobutyl, and tert-butyl groups.

Among the hydroxyalkyl groups, those containing a single hydroxyl group are preferred, and in particular the hydroxyethyl, hydroxypropyl, hydroxybutyl, hydroxypentyl and hydroxyhexyl groups, are preferred.

Among the salts, there may be mentioned in particular the salts of inorganic acids, such as sulphuric acid, hydrochloric acid, hydrobromic acid, hydriodic acid, phosphoric acid, and boric acid. There may also be mentioned the salts of organic adds, which may contain one or more carboxylic, sulphonic phosphonic acid groups. They may be linear, branched, or cyclic aliphatic acids or aromatic acids. These acids may additionally contain one or more heteroatoms chosen from O and N, for example in the form of hydroxyl groups. There may be mentioned in particular propionic acid, acetic acid, terephthalic acid, citric acid, and tartaric acid.

The expression solvate is understood to mean a stoichiometric mixture of the said compound of formula (III) with one or more molecules of water or of organic solvent, such a mixture typically being obtained from the synthesis of the compound of formula (III).

Embodiments of formula (III), there may be mentioned N-(2-hydroxyethyl)urea; N-(2-hydroxypropyl)-urea; N-(3-hydroxypropyl)urea; N-(2,3-dihydroxypropyl)-urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(tris-hydroxymethylmethyl)urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; salts and solvates thereof, and mixtures thereof.

In accordance with the various embodiments total amount of the urea or derivative thereof is in a range from about 20% to about 40%, or from about 20% to about 35%, or from about 25% to about 30%, or from about 25% to about 40%, or from about 30% to about 35% or any suitable combination, sub-combination, range, or sub-range thereof by weight, based on the weight of the composition. One of ordinary skill in the art, however, will appreciate that other ranges are within the scope of the invention.

Thus, the urea or derivative thereof, is present, by weight, based on the total weight of the composition, from about 20, 21, 22, 23, 24, 25, 26, 27, 28, 29, 30, 31, 32, 33, 34, 35, 36, 37, 38, 39 to about 40 weight percent, including increments and ranges therein and there between.

### Amino Acid

In accordance with the various embodiments, the skin conditioning composition includes at least one or more amino acids. In some embodiments the at least one amino acid may be selected from the group consisting of naturally occurring amino acids. In some embodiments the at least one amino acid may be selected from glycine, proline, serine, and cysteine. And in some embodiments the at least one amino acid comprises glycine.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form. Polymeric forms are also useful, such as poly arginine, poly lysine, etc. Additional non-limiting examples include basic amino acids comprising an additional amine function optionally included in a ring or in a ureido function. In some cases, amino acids that may be used include, but are not limited to, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In accordance with the various embodiments, the amount of amino acid present in a composition according to the disclosure can range from about 2% to about 10%, or from about 3% to about 9%, or from about 4% to about 8%, or from about 3% to about 5%, or any suitable combination, sub-combination, range, or sub-range thereof by weight, based on the weight of the composition. In some embodiments according to the disclosure, the composition includes at least about 3%, or at least about 5% of the at least one amino acid. In some embodiments according to the disclosure, the composition includes not more than about 9% of the at least one amino acid. One of ordinary skill in the art, however, will appreciate that other ranges are within the scope of the invention. In some embodiments, two or more alkaline boosters are present.

Thus, any one of or a combination of the amino acid may be present, by weight, based on the total weight of the composition, or from about 2.0, 2.5, 3.0, 3.5, 4.0, 4.5, 5.0, 5.5, 6.0, 6.5, 7.0, 7.5, 8.0, 8.5, 9.0, 9.5 to about 10.0 weight percent, including increments and ranges therein and there between.

### Triacetin/Triethyl Citrate

In accordance with the various embodiments, the skin conditioning composition includes at least one or more of triacetin or triethyl citrate.

In accordance with the various embodiments, the amount of triacetin or triethyl citrate present in a composition according to the disclosure can range from about 0.2% to about 2%, or from about 0.3% to about 1.5%, or from about 0.5% to about 1.2%, or from about 0.7% to about 1%, or any suitable combination, sub-combination, range, or sub-range thereof by weight, based on the weight of the skin conditioning composition. In some embodiments according to the disclosure, the skin conditioning composition includes at least about 0.2%, or at least about 0.7% of the triacetin or triethyl citrate. In some embodiments according to the disclosure, the skin conditioning composition includes not more than about 1% of the triacetin or triethyl citrate. One of ordinary skill in the art, however, will appreciate that other ranges are within the scope of the invention. In some embodiments, two or more alkaline boosters are present.

Thus, any one of or a combination of the triacetin or triethyl citrate may be present, by weight, based on the total weight of the skin conditioning composition, or from about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, to about 2 weight percent, including increments and ranges therein and there between.

### Buffer System

In accordance with the various embodiments, the skin conditioning composition may include a buffer system. In some embodiments, the buffer system comprises one or a combination of components selected from the group consisting of citric acid, sodium citrate, sodium lactate, lactic acid, and combinations thereof. In some particular embodiments, the buffer system comprises sodium lactate, lactic acid, ammonium lactate, and combinations thereof. In some particular embodiments, the buffer system comprises citric acid, sodium citrate, and combinations thereof.

More generally, the buffer system is selected from conventional buffer systems commonly used for compositions used on keratinous tissue, particularly skin. Buffer systems may include one or a combination of acidifying agents and alkalifying agents. Acidifying agents include, for example, organic or inorganic acids such as hydrochloric acid, orthophosphoric acid, sulphuric acid, carboxylic acids such as acetic acid, tartaric acid, citric acid and lactic acid, and sulphonic acids. Alkalifying agents include, for example, aqueous ammonia, alkali metal carbonates, alkanolamines such as mono-, di- and triethanolamines and their derivatives, sodium hydroxide or potassium hydroxide.

In accordance with the various embodiments, the amount of buffer components, when present in a skin conditioning composition according to the disclosure, generally conform to those employed conventionally. According to the disclosure, a buffer system comprising, for example, sodium lactate, lactic acid, ammonium lactate, and combinations thereof, includes each of the buffer system components present in a range from about 0.1% to about 3%, or from about 0.25% to about 2.5%, or from a total of about 0.1% to about 6%, or from about 0.25% to about 5.25%, or any suitable combination, sub-combination, range, or sub-range thereof by weight, based on the weight of the skin conditioning composition. More generally, in some embodiments according to the disclosure, the skin conditioning composition includes each one or more buffer components present in a range from about 0.1% to about 15%. One of ordinary skill in the art, however, will appreciate that other ranges are within the scope of the invention. In some embodiments, two or more alkaline boosters are present.

Thus, any one of or a combination of buffer system components may be present, by weight, based on the total weight of the skin conditioning composition, or from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, to about 15 weight percent, including increments and ranges therein and there between.

### Glycol

In accordance with the various embodiments, the skin conditioning composition may include at least one alkanol, and particularly at least one glycol. In some embodiments, the cosmetic skin conditioning comprises one or a combination of alkanols (polyhydric alcohols such as glycols and polyols). In some embodiments, the cosmetic skin conditioning comprises one or a combination of propylene glycol, glycerin, propanediol, butylene glycol, dipropylene glycol, pentylene glycol, hexylene glycol, ethoxydiglycol, and combinations thereof. In some particular embodiments, the cosmetic skin conditioning may comprise at least propylene glycol, with or without glycerin.

More generally, the cosmetic skin conditioning may comprise one or a combination of the 1,2,6-hexanetriol, trimethylolpropane, ethylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments, the amount of the at least one glycol present in the skin conditioning composition can range from about 1% to about 15%, or from about 2% to about 12%, or from about 3% to about 10%, or from about 3.5% to about 5%, or any suitable combination, sub-combination, range, or sub-range thereof by weight, based on the weight of the skin conditioning composition. One of ordinary skill in the art, however, will appreciate that other ranges are within the scope of the invention.

Thus, any one of or a combination of glycol, when present, may be present, by weight, based on the total weight of the skin conditioning composition, is from about 1, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5 13, 13.5, 14, 14.5, to about 15 percent, by weight, based on the weight of the skin conditioning composition, including increments and ranges therein and there between.

### Carrier System

In various embodiments, the skin conditioning composition is formed by providing a carrier system having a water-phase that comprises water. The urea, amino acid, triacetin/Triethyl citrate, and buffer system components are typically preferentially in the water phase. As described herein below, the skin conditioning composition may include other ingredients, and the carrier system may include other phases, including one or more additional water and/or oil phases.

### Water Phase/Water

In various embodiments, the cosmetic skin condition composition includes at least one water phase that includes water.

The skin conditioning composition comprises from about 40% to about 70% by weight of water, with respect to the total weight of the skin conditioning composition. In some embodiments, the amount of water in the skin conditioning composition can range from about 40% to about 70%, or from about 45% to about 65%, or from about 50% to about 60%, or any suitable combination, sub-combination, range, or sub-range thereof by weight, based on the weight of the skin conditioning composition.

The pH of the skin conditioning composition is in the range from and including about 4 to about 6, and in some embodiments is one of between 5.5 and 6.5, or between 4 and 5, or between 5 and 6, or up to about 7. The pH can be adjusted to the desired value by addition of a base (organic or inorganic) to the skin conditioning composition, for example ammonia or a primary, secondary, or tertiary (poly)amine, such as monoethanolamine, diethanolamine, triethanolamine, isopropanolamine or 1,3-propanediamine, or alternatively by addition of an inorganic or organic acid, for example a carboxylic acid, such as, for example, citric acid.

Thus, water may be present by weight, based on the total weight of the skin conditioning composition, or from about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70 weight percent, including increments and ranges therein and there between.

### Optional Components

### Cosmetic Oils/Emollients

In accordance with some embodiments, the skin conditioning composition may comprise at least one oily phase which may include any one or a combination of cosmetic oils. In some embodiments, the oil is generally immiscible in water. The oil may be selected from hydrocarbons, silicones, fatty alcohols, glycols, and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and combinations thereof.

In some particular embodiments, the oil may be selected from the group consisting of, coco-caprylate/caprate, dicaprylyl carbonate, dicaprylyl ether, isopropyl palmitate, and combinations thereof.

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil, or a synthetic hydrocarbon-based oil. Further, suitable oil may be a mineral hydrocarbon-based oil, a plant hydrocarbon-based oil, or a synthetic hydrocarbon-based oil.

### Silicone Oils

The skin conditioning composition may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the skin conditioning composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

### Fluoro Oils

The skin conditioning composition may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

### Hydrocarbon-Based Oils

The skin conditioning composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the skin conditioning composition may include a synthetic oil selected from the group consisting of hydrogenated polyisobutene and hydrogenated polydecene. A hydrocarbon-based oil may be a non-volatile hydrocarbon-based, such as:

(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, rhea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

(ii) synthetic ethers containing from 10 to 40 carbon atoms;

(iii) linear or branched hydrocarbons of mineral or synthetic origin, such as petroleum jelly, polydecenes, hydrogenated polyisobutene such as Parleam, and 40 squalane;

(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;

(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

(vi) higher fatty acids, such as oleic acid, linoleic acid, or linolenic acid;

(vii) carbonates, such as dicaprylyl carbonate, such as the product sold under the name Cetiol CC™ by Cognis;

(viii) fatty amides, such as isopropyl N-lauroyl sarcosinate, such as the product sold under the trade name Eldew SL 205™ from Ajinomoto; and

(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the skin conditioning composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the skin conditioning composition may comprise one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol % to about 350 g/mol.

In some embodiments, the skin conditioning composition may comprise polar emollients that include those derived from C12-C50 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

The one or more oil, when present, alone or in combination as a blend of oils, may be present in the skin conditioning composition from about 0.0001% to about 20% or from about 0.001% to about 0.0010%, or from about 0.003% to about 0.004%, or from about 0.01% to about 0.1%, or from about 0.1% to about 10%, or from about 0.5% to about 20%, or from about 1% to about 10%, or from about 5% to about 10%, or from about 2% to about 7%, or from about 0.5% to about 2%, or from about 0.008% to about 0.01%, or from about 0.1% to about 0.2%, or from about 0.5% to about 2%, or from about or any suitable combination, sub-combination, range, or sub-range thereof by weight, based on the weight of the skin conditioning composition. One of ordinary skill in the art, however, will appreciate that other ranges are within the scope of the invention. In some embodiments, the skin conditioning composition includes more than one oil, each oil present in an amount as set forth herein above, wherein each different oil (such as, for example, plant oils and extracts with oils) may be present within one of the ranges selected from the ranges set forth herein above.

Thus, each of the at least one oil or combination of oils, if present, is present by weight, based on the total weight of the skin conditioning composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19 to about 20 percent, including increments and ranges therein and there between.

### Surfactant

In accordance with the disclosure, various non-limiting embodiments of the skin conditioning composition may optionally include at least one surfactant. And in accordance with the disclosure, various non-limiting embodiments of the skin conditioning composition that include an oil, for example, but not limited to, a Vitamin E component, such as tocopherol, may optionally include at least one surfactant. In some embodiments, the skin conditioning composition comprises no oil. In some embodiments, the skin conditioning composition comprises no surfactant. In some embodiments, the skin conditioning composition comprises at least one oil, for example, tocopherol, with at least one surfactant. In some embodiments, the skin conditioning composition comprises at least one oil with more than one surfactant. In some embodiments, the skin conditioning composition comprises at least one oil without any surfactants, wherein the total amount of oil present does not inhibit forming the skin conditioning composition as a clear, single phase solution.

The at least one surfactant may be an nonionic, cationic, anionic, or a zwitterionic surfactant. The at least one surfactant may be selected from the group consisting of polyoxyethylene sorbitan monolaureate, laureth-23, polyoxyethylated octyl phenol, 3-((3-cholamidopropyl) dimethylammonio)-1 propane sulfonate, sodium dilauramidoglutamide lysine, cholate, deoxycholate, sodium dodecylsulfate, TWEEN-80, and combinations thereof. The at least one surfactant may exclude esters. The at least one surfactant may be present in the skin conditioning composition in a range from about 1% to about 5%, based on the weight of the skin conditioning composition.

In some particular embodiments the at least one surfactant, when present, comprises one or a combination of polyoxyethylene sorbitan monolaureate and laureth-23.

In various embodiments, the at least one surfactant, when present, may be present from about 1% to about 5% by weight of the skin conditioning composition, and in some embodiments, from about 1% to about 2%, and in some embodiments, from about 3% to about 6%, and in some embodiments, from about 3% to about 4.5%, or any suitable combination, sub-combination, range, or sub-range thereof by weight, based on the weight of the skin conditioning composition. In some embodiments, laureth-23 may be present from about 1% to about 5% by weight of the skin conditioning composition, and in some embodiments, from about 3% to about 4.5%, or any suitable combination, sub-combination, range, or sub-range thereof by weight, based on the weight of the skin conditioning composition. In some embodiments, polyoxyethylene sorbitan monolaureate may be present from about 1% to about 5% by weight of the skin conditioning composition, and in some embodiments, from about 1% to about 2%, or any suitable combination, sub-combination, range, or sub-range thereof by weight, based on the weight of the skin conditioning composition. In some embodiments, the skin conditioning composition comprises more than one surfactant. One of ordinary skill in the art, however, will appreciate that other ranges are within the scope of the invention.

Thus, in various embodiments, when present, each of the at least one surfactant may be present in a composition according to the disclosure from about 1, 2, 3, 4, to about 5 percent, by weight, including increments and ranges therein and there between.

Polymeric Thickener

In accordance with the disclosure, various non-limiting embodiments of the skin conditioning composition may optionally include one or more polymeric thickeners. In some embodiments, the one or more thickener may be selected from one or more of natural gums and synthetic polymers, for example, the thickener may be selected from the group consisting of starches (corn, rice, tapioca, potato), gums (xanthan carrageenan, gellan, sclerotium, tarabiotech fermentation). In some particular embodiments, the thickener may be selected from acrylates/C10-30 alkyl acrylate crosspolymer, carbomer, xanthan gum, hydroxypropyl guar, ceratonia siliqua (carob) gum, ammonium polyacryloyldimethyl taurate, ammonium acryloyldimethyltaurate/steareth-25 methacrylate crosspolymer, and polyacrylate crosspolymer-6.

In some embodiments, the polymeric thickener may be one of carbomer, acrylates/C10-30 alkyl acrylate crosspolymer, polyacrylate crosspolymer-6, microcrystalline cellulose (and) cellulose gum, xanthan gum, sodium carboxymethyl starch, sclerotium gum (and) xanthan gum, xanthan gum (and) ceratonia siliqua (carob) gum (50/50), dehydroxanthan gum, hydroxypropyl starch phosphate, sclerotium gum (and) xanthan gum (75/25), sclerotium gum, xanthan gum (and) sclerotium gum (and) Lecithin (and) pullulan, or combinations thereof.

In some embodiments the skin conditioning composition may comprise two or more polymeric thickeners. The amount of each of the at least one polymeric thickener, when present, may be present in the skin conditioning composition in a range of from about 0.01% to about 2%, or from about 0.01% to about 1.5%, or from about 0.3% to about 1.2%, or any suitable combination, sub-combination, range, or sub-range thereof by weight, based on the weight of the skin conditioning composition. One of ordinary skill in the art, however, will appreciate that other ranges are within the scope of the invention.

In some embodiments, the total amount of polymeric thickener in the skin conditioning composition, when present, is present from about 0.01% to about 5%, or from about 0.02% to about 2%, or from about 0.03% to about 1.5%, or from about 0.1% to about 0.2%, or any suitable combination, sub-combination, range, or sub-range thereof by weight, based on the weight of the skin conditioning composition. One of ordinary skill in the art, however, will appreciate that other ranges are within the scope of the invention.

Thus, one or more polymeric thickener, when present, is present by weight, based on the total weight of the skin conditioning composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, including increments and ranges therein and there between.

### Preservatives

In accordance with the disclosure, various non-limiting embodiments of the skin conditioning composition may optionally include one or more preservatives and/or antimicrobials. Any preservative commonly used in cosmetic formulations is an acceptable preservative for the skin conditioning compositions herein, such as phenoxyethanol, salicylic acid, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or for example.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the skin conditioning compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

Other suitable preservatives include, but are not limited to, chloroacetamide, triclosan and iodopropynyl butylcarbamate, pyridine derivatives (e.g., pyrithione and zinc pyrithione), chlorphenesin, phenyl mercuric salts, phenoxyethanol, and other known preservative systems.

In some embodiments, the preservative includes one or more preservatives, the one or combination present at a concentration, by weight of about 0.001% to about 5%, or alternatively about 0.05% to about 2.5% or alternatively about 0.1% to about 2.0%, based upon weight of the skin conditioning composition.

Thus, any one of or a combination of preservatives, when present, may be present, by weight, based on the total weight of the skin conditioning composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

### Other Optional Components

In accordance with the disclosure, various non-limiting embodiments of the skin conditioning composition may optionally include one or more other additives or actives (optional components), selected from, for example, actives such as acetyl trifluoromethylphenyl valylglycine; a humectant in addition to any glycol, for example caprylyl glycol, squalane, sugars, such as, glucose, xylitol, maltitol, sorbitol, sucrose pentaerythritol, inositol, pyrrolidone carboxylic acid, lactic acid, lithium chloride, acetamide MEA, dicyanamide, hyaluronic acid, aloe vera, honey, and seaweed extract; preservative and anti-microbial components (such as those listed above), particularly, for example, salicylic acid, and phenoxyethanol; phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; and vitamins and vitamin derivatives, such as calcium pantothenate, tocopherol and ascorbic acid; fillers such as clays, talc, organic thickeners with for instance, anionic, cationic, nonionic, and amphoteric polymeric associative thickeners and combinations thereof; penetrants; sequestrants; fragrances; dispersants; film-forming agents; ceramides; opacifiers; organic or inorganic sunscreen actives and SPF boosters; and combinations thereof.

Although the aforementioned optional components are given as an example, it will be appreciated that other optional components compatible with cosmetic applications known in the art may be used.

In some embodiments, one or more optional components, alone or in combination, can be present in the skin conditioning composition according to the disclosure from about 0.05% to about 50% by weight, or from about 1% to about 30%, or from about 1.5% to about 20%, and from about 5% to about 15%, or from about 0.05% to about 2.5% by weight, or from about 0.1% to about 2%, or from about 0.25% to about 5%, and from about 0.5% to about 10%, or any suitable combination, sub-combination, range, or sub-range thereof by weight, based on the weight of the skin conditioning composition.

In some embodiments, one or more other components, such as preservatives, vitamins, and the like, alone or in combination, can be present in the skin conditioning composition according to the disclosure from about 0.05 to about 50% by weight, or from about 0.05% to about 25% by weight, or from about 0.1 to about 10%, or from about 0.25% to about 5%, and from about 0.5 to about 3.5%, or any suitable combination, sub-combination, range, or sub-range thereof by weight, based on the weight of the skin conditioning composition.

In some exemplary embodiments, preservatives may include salicylic acid, and phenoxyethanol.

Thus, one or a combination of optional components may be present, by weight, based on the total weight of the skin conditioning composition, or from about 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### Complementary Compositions

In various embodiments, complementary compositions may include any one or more of the ingredients described herein above, and in some examples specifically as described in the Optional Components.

### Desquamating Agents/Hydroxy Acids

In accordance with the various embodiments of methods of use of the skin conditioning composition, the methods of use may include use of a complementary peel composition comprising at least one active ingredient selected from desquamating agents, for example, hydroxy acids.

### Alpha Hydroxy Acid

In accordance with the various embodiments, the chemical peel component of the skin-enhancing treatment comprises at least one alpha hydroxy acid.

Suitable alpha hydroxy acids include glycolic acid, lactic acid, tartaric acid, mandelic acid, citric acid, ester derivatives thereof and combinations thereof. Exemplary ester derivatives include ester compounds of lactic acid, such as methyl lactate, ethyl lactate, butyl lactate and, similarly, ester compounds of glycolic acid, tartaric acid, mandelic acid, citric acid. One particularly suitable alpha hydroxy acid is lactic acid. Lactic acid, or 2-hydroxypropanoic acid, is provided to the chemical peel composition to provide enhanced exfoliation of the skin. In addition, lactic acid also boosts production of glycosaminoglycan (GAG) in the skin, improving the barrier function and moisturization of skin.

And in some particular embodiments, the alpha hydroxy acid in the chemical peel component of the skin-enhancing treatment excludes at least one of lactic acid, glycolic acid, tartaric acid, mandelic acid, and citric acid.

The chemical peel component of the skin-enhancing treatment includes a concentration of alpha hydroxy acid in a range from about 4% to about 15%, or from about 8% to about 14% or from about 9% to about 13%, or from about 10% to about 12%, or is about 10%, or any suitable combination, sub-combination, range, or sub-range thereof by weight, based on the weight of the chemical peel component of the skin-enhancing treatment. One of ordinary skill in the art, however, will appreciate that other ranges are within the scope of the invention. In accordance with some embodiments, the amount of alpha hydroxy acid present is not more than about 10%.

Thus, any one of or a combination of alpha hydroxy acid is present, by weight, based on the total weight of the chemical peel component of the skin-enhancing treatment, from about 4, 5, 6, 7, 8, 9, 10, 11, 12, 13, 14, to about 15 weight percent, including increments and ranges therein and there between.

### Beta Hydroxy Acid

In accordance with the various embodiments, the chemical peel component of the skin-enhancing treatment comprises at least one beta hydroxy acid. In some particular embodiments, the chemical peel component of the skin-enhancing treatment includes a beta hydroxy acid comprising salicylic acid.

The term “beta-hydroxy acid” is understood to mean, according to the present invention, a carboxylic acid having a hydroxyl functional group and a carboxylic functional group separated by two carbon atoms. A beta hydroxy acid can be present in the chemical peel component of the skin-enhancing treatment in the form of the free acid and/or in the form of one of its associated salts (salts with an organic base or an alkali metal, in particular), especially according to the final pH imposed on the chemical peel component of the skin-enhancing treatment.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

And in some particular embodiments, the beta hydroxy acid in the chemical peel component of the skin-enhancing treatment excludes at least one of salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid.

The chemical peel component of the skin-enhancing treatment includes a concentration of beta hydroxy acid in a range from about 0.1% up to and not more than about 2% of beta hydroxy acid, by weight, based on the total weight of the chemical peel component of the skin-enhancing treatment. In some embodiments, the chemical peel component of the skin-enhancing treatment includes up to and not more than about 2%, or about 1.9% of beta hydroxy acid. In some embodiments, the chemical peel component of the skin-enhancing treatment includes up to and not more than about 1% of beta hydroxy acid. In accordance with some embodiments, the amount of beta hydroxy acid present is not more than about 0.40% to about 0.50%. In some embodiments, the chemical peel component of the skin-enhancing treatment includes from about 0.1% to about 1% of beta hydroxy acid, or from about 0.2% to about 2.0%, or from about 0.1% to about 1.5%, or from about 0.2% to about 1.5%, or from about 0.3% to about 1.0%, or from about 0.35% to about 0.75%, or from about 0.4% to about 0.5%, or is about 0.45%, or any suitable combination, sub-combination, range, or sub-range thereof by weight, based on the weight of the chemical peel component of the skin-enhancing treatment. One of ordinary skill in the art, however, will appreciate that other ranges are within the scope of the invention.

Thus, the at least one beta hydroxy acid is present in the chemical peel component of the skin-enhancing treatment from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

### Phenylethyl Resorcinol

In accordance with the various embodiments of methods of use of the skin conditioning composition, the methods of use may include use of a complementary composition comprising phenylethyl resorcinol.

Phenylethyl resorcinol functions a tyrosinase inhibitor. The phenylethyl resorcinol, when utilized in the chemical peel component of the skin-enhancing treatment effectively whiten skin and reduce skin tone unevenness. Phenylethyl resorcinol has not shown adverse effects in basic toxicological tests, including acute oral toxicity, mutagenicity, skin irritation, skin sensitization, and phototoxicity.

In accordance with the various embodiments, when present, the amount of phenylethyl resorcinol present in the chemical peel component of the skin-enhancing treatment is from about 0.2% to about 2.0%, or from about 0.2% to about 1.5%, or from about 0.4% to about 1.0%, or from about 0.6% to about 0.9%, or from about 0.7% to about 0.8%, or from about 0.2% to about 0.8%, is about 0.75%, or any suitable combination, sub-combination, range, or sub-range thereof by weight, based on the weight of the chemical peel component of the skin-enhancing treatment. One of ordinary skill in the art, however, will appreciate that other ranges are within the scope of the invention. In accordance with some embodiments, the amount of phenylethyl resorcinol present is not more than about 0.2% to about 0.8%.

Thus, phenylethyl resorcinol, when present in the chemical peel component of the skin-enhancing treatment, is present, by weight, based on the total weight of the chemical peel component of the skin-enhancing treatment, from about 0.20, 0.30, 0.40, 0.50, 0.60, 0.70, 0.80, 0.90, 1.0, 1.1, 1.2, 1.3, 1.4, 1.5, 1.6, 1.7, 1.8, 1.9 to about 2.0 weight percent, including increments and ranges therein and there between.

### Articles of Manufacture

In accordance with the various embodiments, the skin conditioning composition may be provided in a kit or other article of manufacture.

It will be appreciated that in the various embodiments of packaging, that the sub compositions that are packaged are maintained as separate until the skin conditioning composition and the at least one complementary composition is intended to be applied to keratinous tissue, at which time they may be separately applied or may be applied together, or may be combined and mixed prior to application.

Generally, the skin conditioning composition is separate from the complementary composition until the skin conditioning composition is intended to be applied to keratinous tissue. Thus, one sub composition includes at least the hydrogen peroxide and any additional optional oxidizing agent, and one sub composition includes at least the alkaline booster. Variously, each of the sub compositions may include any one or more of the fatty compounds and any one or more of solvents, surfactants, polymers, actives, and other components. Further, any one or more of the fatty compounds and any one or more of solvents, actives and other components may be separately packaged from the other sub compositions, such that the article of manufacture may comprise more than two separate packages or package chambers.

In one embodiment, the article of manufacture may be a packet with separate packages or chambers separated by a frangible seal between the chambers. In use, the seal is broken by the user to contact the separately packaged sub compositions and mix them prior to application onto the skin. In yet other embodiments, the article of manufacture may be a tube with dual chambers. Two parts are mixed when squeezing out from each chamber. In yet other embodiments, the article of manufacture may be a dual-chamber pump and the two parts can be mixed after pumping out of the container. In some other embodiments, the packaging is a single tube with a seal between the two components that is broken to mix. In other embodiments, the packaging can be a single container holding a suspension of encapsulated material that is mixed/broken to disperse and mix. And in yet other embodiments, the packaging is otherwise sufficient to retain the skin conditioning composition separate from the complementary composition until the skin conditioning composition is intended to be applied to keratinous tissue.

In some embodiments, the one or both of the first and second parts may be delivered by spray application, or other applicator selected from a pump, or brush.

In accordance with the various embodiments, a composition of the system for forming a continuous film mask is in a form including a suspension, lotion, cream, serum, essence, gel, stick, spray, ointment, paste, foam, mousse, cream, wipe, patch, strip, film-forming product.

### EXAMPLES

### Example 1: Raw Materials

The examples below according to the invention are given by way of illustration and without a limiting nature. The names are the chemical name or the INCI name. The amounts of active ingredients are given therein as % by weight based on raw material compositions comprising 100% of the listed ingredient, unless otherwise mentioned. Compositions and skin rejuvenation systems as described in the representative embodiments herein are selected from commercially available materials, including, as shown in the exemplified embodiments herein below, for example: Urea, glycine, serine, Latic acid, sodium lactate, triacetin, and triethyl citrate.

TABLE 2
Raw Materials

| | PERCENT ACTIVE |
|---|---|
| RM INGREDIENT | (IF < 100%) |
| Urea | |
| Glycine | |
| Serine | |
| Latic Acid | 90 |
| Sodium Lactate | 60 |
| Triacetin | |
| Triethyl Citrate | |

### Example 2: Inventive Compositions and Skin Rejuvenation Systems

Five exemplary embodiments of the inventive composition are shown in Table 3.

TABLE 3
Inventive Composition

| | INV | INV | INV | INV | INV |
|---|---|---|---|---|---|
| INGREDIENT | #1 | #2 | #3 | #4 | #5 |
| UREA | 40 | 40 | 30 | 30 | 30 |
| SODIUM LACTATE | 2.5 | 2.5 | 2.5 | 2.5 | 2.5 |
| SALICYLIC ACID | 0.45 | 0.45 | 0.45 | 0.45 | 0.45 |
| LACTIC ACID | 0.5 | 0.5 | 0.5 | 0.5 | 0.5 |
| AMMONIUM LACTATE | | 2.5 | | | |
| TRIACETIN | 0.4 | 0.7 | 0.7 | 0.7 | 0.7 |
| GLYCINE | 5 | 5 | 5 | 5 | 5 |
| ACETYL TRIFLUORO- | | | | | 2 |
| METHYLPHENYL | | | | | |
| VALYLGLYCINE | | | | | |
| PROPYLENE GLYCOL | 7 | 4.5 | 7 | 7 | 7 |
| GLYCERIN | | | | 3.5 | 3 |
| CAPRYLYL GLYCOL | | | 0.3 | 0.3 | 0.3 |
| TRIETHANOLAMINE | 0.9 | 0.9 | 0.9 | 0.9 | 0.9 |
| PHENOXYETHANOL | | 0.7 | 0.7 | 0.7 | 0.7 |
| AQUA | 43.25 | 42.25 | 51.95 | 48.45 | 46.95 |

### Example 3: Effect of Urea and Hydroxyethyl Urea on Peel Efficacy Using a Hydroxy Acid

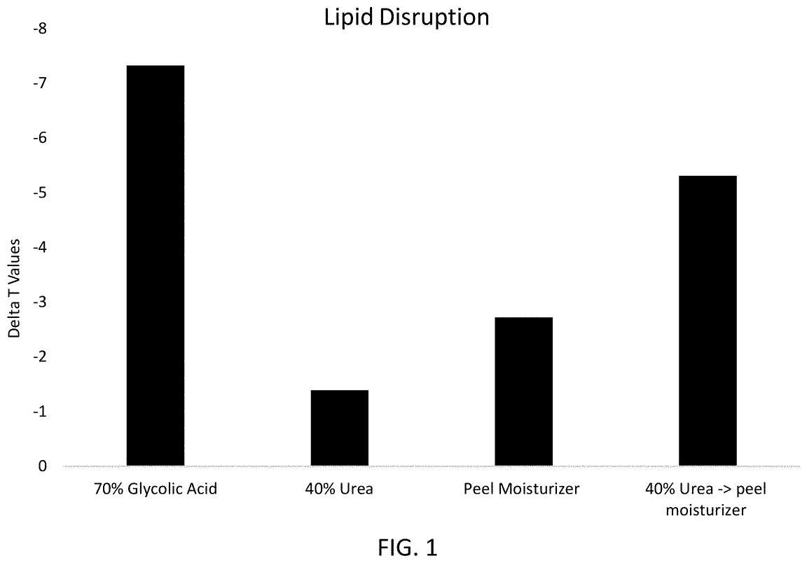

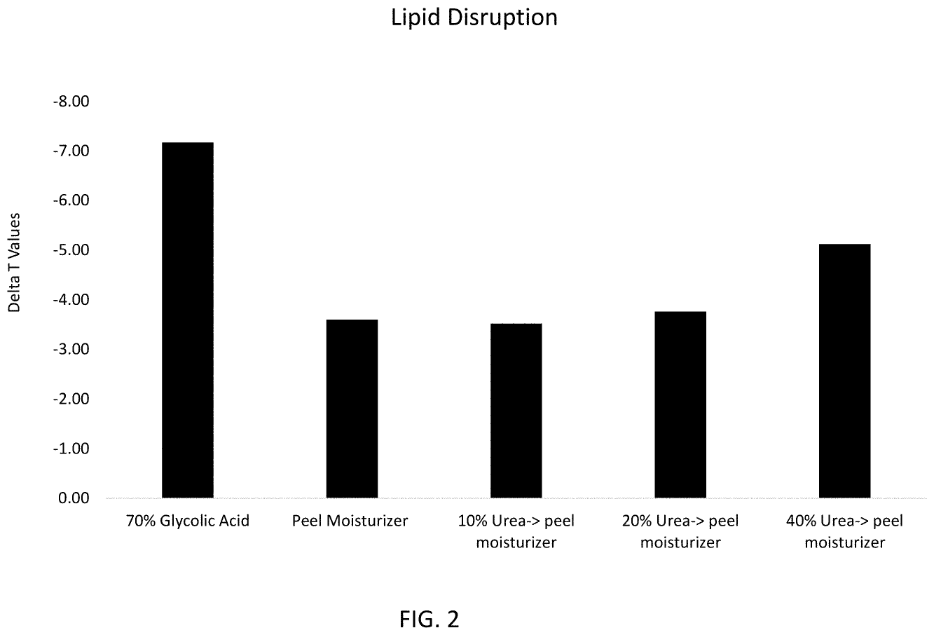

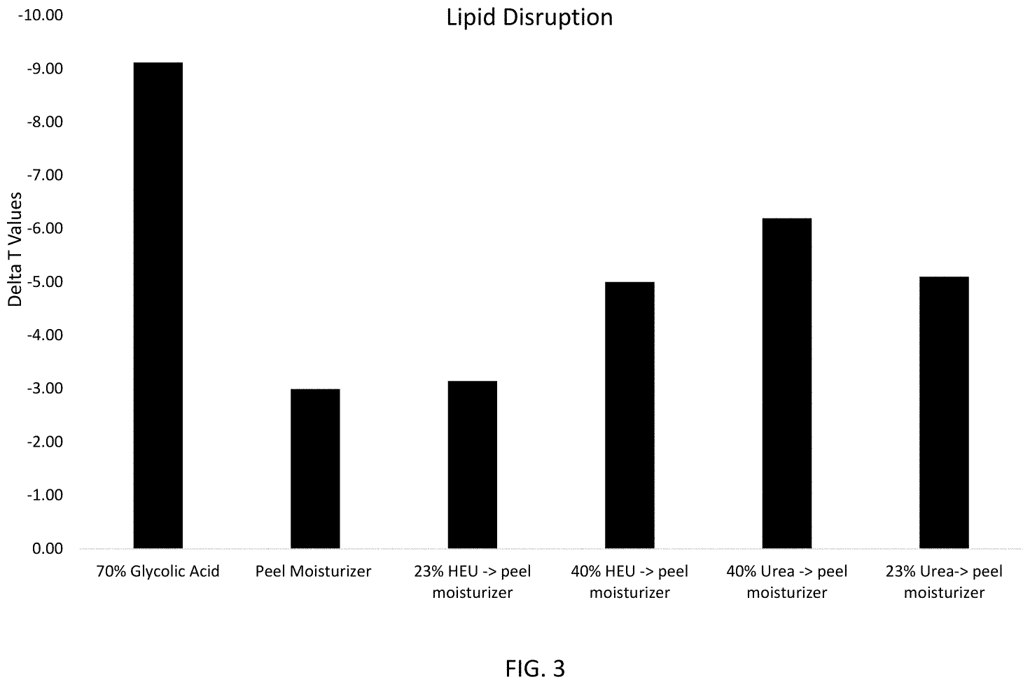

In studies, the effect of urea at various concentrations and using hydroxyethyl urea (HEU) was according to INV #1 (40% urea, 5% glycine, 0.7% triacetin, buffer) in which the amount and form of urea was varied. These results were compared with controls that include glycolic acid as a positive benchmark control, a peel composition without use of the inventive composition, and the variations on INV #1. Studies were conducted with each of the tested compositions to evaluate effect of the compositions on the stratum corneum. The results are shown in FIG. 1, FIG. 2 and FIG. 3.

In Vitro Stratum Corneum Comparison of Δ T using differential scanning calorimetry (DSC) is used to characterize the stability of a protein or other biomolecule directly in its native form. It does this by measuring the heat change associated with the molecule's thermal denaturation when heated at a constant rate. Compositions were evaluated on SC samples according to the following procedure. Stratum corneum samples were equilibrated at 75% relative humidity (RH) overnight. Inventive and Comparative Compositions and control treatments applied on SC surface (0.1 g/sample) for 15 min in 75% RH. The SC surface was wiped clean and rinsed with DI water. Allow to dry in 75% RH overnight. The DSC profile was measured from 20° C. to 120° C. at 5° C./min.

As an initial step, each test composition was applied first to skin without rinsing, followed by application of a hydroxy-acid based peel composition in a moisturizer form. The studies revealed that increased amounts of each of urea and hydroxyethyl urea resulted in increased desquamation as compared to use of the peel moisture alone, the desquamation evidenced by stratum corneum disorganization. Surprisingly, the compositions with urea outperformed those with HEU, and in particular, the 23% urea form performed as well as the 40% HEU form.

While illustrative embodiments have been illustrated and described, it will be appreciated that various changes can be made therein without departing from the spirit and scope of the invention.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step or material other than those specified in the claim and, in the latter instance, impurities ordinarily associated with the specified material(s). The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.” Further, it should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. The term “about” means plus or minus 5% of the stated value.

Other than in the operating examples, or where otherwise indicated, all numbers expressing quantities of ingredients and/or reaction conditions modified by the term “about” are to be understood as being within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A method for improving the efficacy of a skin care active, said method comprising:
applying to a region skin, in any order:
a first composition comprising a skin conditioning composition comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH is less than about 7; and
a second composition comprising a complementary composition comprising one or more skin actives not present in the skin conditioning composition.

2. The method according to claim 1, wherein each of the compositions is aqueous and is applied in liquid form.

3. The method according to claim 1, wherein the first composition is one of a water based toner or a mask.

4. The method according to claim 1, wherein the first composition is a water based toner that is applied first followed by the second composition without prior removal of the first composition.

5. The method according to claim 1, wherein the first composition is a water based mask that is applied first followed by the second composition, the method further comprising a step that includes rinsing to remove the first composition prior to application of the second composition.

6. The method according to claim 1, wherein one or both of the first and second compositions is coupled to an application surface of an applicator, the application surface selected from the group consisting of a bristle surface of a brush, a roller surface of a rolling applicator, and a blade surface of a spatula.

7. The method according to claim 1, said method comprising the steps of:
a) applying to a region of skin the first composition;
b) applying to the region of skin the second composition.

8. A skin rejuvenation system, comprising:
a) A skin conditioning composition comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH is less than about 7; and
b) a complementary composition comprising one or more skin actives not present in the skin conditioning composition.

9. The skin rejuvenation system according to claim 8, wherein the skin conditioning composition is formulated as a leave-on product or a rinse of product.

10. The skin rejuvenation system according to claim 8, wherein, respecting the complementary composition, the composition is a leave on or rinse of skin peel composition comprising at least a hydroxy acid.

11. The skin rejuvenation system according to claim 8, wherein, respecting the skin conditioning composition, the pH is in a range from about 4.0 to about 6.0.

12. The skin rejuvenation system according to claim 8, wherein, respecting the skin conditioning composition, the at least one amino acid is soluble in water at a pH from about 4 to about 6.

13. The skin rejuvenation system according to claim 8, wherein, respecting the skin conditioning composition, the at least one amino acid is selected from the group consisting of glycine, proline, serine, cysteine and combinations thereof.

14. The skin rejuvenation system according to claim 8, wherein, respecting the skin conditioning composition, the at least one amino acid is present in an amount from about 5% to about 9%, by weight, based on the weight of the composition.

15. The skin rejuvenation system according to claim 8, wherein, respecting the skin conditioning composition, the at least one of triacetin or triethyl citrate is present in an amount from about 0.2% to about 2%, by weight, based on the weight of the composition.

16. The skin rejuvenation system according to claim 8, wherein, respecting the skin conditioning composition,
a) the urea or a derivative thereof, is present in an amount from greater than about 20% to about 40%, by weight, based on the weight of the composition, and further comprising at least one glycol selected from the group consisting of propylene glycol, glycerin, and combinations thereof;
b) the at least one amino acid the at least one amino acid is soluble in water at a pH from about 4 to about 6 and is selected from the group consisting of glycine, proline, serine, cysteine and combinations thereof and is present in an amount from about 5% to about 9%, by weight, based on the weight of the composition;
c) the at least one of triacetin or triethyl citrate, is present in an amount a range that includes about 0.2% to about 2%, by weight, based on the weight of the composition; and
d) wherein the skin conditioning composition comprises a buffer system comprising one or a combination of components selected from the group consisting of citric acid, sodium citrate, sodium lactate, lactic acid, and combinations thereof;
e) wherein the pH of the skin conditioning composition is in a range from about 4.0 to about 6.0 and the skin conditioning composition optionally comprises at least one pH adjusting reagent comprising triethanolamine; and
f) wherein the skin conditioning composition optionally comprises propylene glycol and excludes glycerin,
wherein the skin conditioning composition demonstrates structural and pH stability for periods of up to about 8 weeks under temperature conditions that range from 4° C. up to 45° C. and through freeze thaw cycles from −20° C. through 20° C., characterized as the absence of any or sustained visible crystallization and the stabilization of pH at or under a pH of about 7.

17. A article of manufacture for providing a skin system for improving the efficacy of a skin care active, the article of manufacture comprising:
a) at least first and second separately contained compositions
i) the first composition comprising a skin conditioning composition comprising: a carrier system comprising a water phase, the water phase comprising urea or a derivative thereof, present in an amount from about 20% to about 40%, by weight, based on the weight of the composition; at least one amino acid; at least one of triacetin or triethyl citrate; and, optionally, a buffer system having a pH in a range from about 4.0 to about 6.0, wherein the pH is less than about 7, and
ii) the second composition comprising complementary composition comprising one or more skin actives not present in the skin conditioning composition.

18. The article of manufacture according to claim 19, wherein the first and second compositions are one of (1) contained to be mixable prior to application to skin, (2) contained to be applied separately, in any order, (3) are each coupled to an application surface of an applicator, or (4) are each separately disposed in a first compartment of a sachet and the application surface may be a surface of a second compartment of the sachet, and wherein the first compartment and the second compartment are fluidically separated by a breakable barrier.

19. The article of manufacture according to claim 19, wherein the application surface may be a surface of a mask configured to conform to a face of a wearer

20. The article of manufacture according to claim 19, wherein the application surface may be selected from the group consisting of a bristle surface of a brush, a roller surface of a rolling applicator, and a blade surface of a spatula.
